(19) United States
(12) Patent Application Publication
(10) Pub. No.: US 2023/0313033 A1
(43) Pub. Date: Oct. 5, 2023

(54) Nanoparticles To Improve Analytical Signal

(71) Applicant: Alma Mater Studiorum Università di Bologna, Bologna (IT)

(72) Inventors: Luca Prodi, Bologna (IT); Francesco Paolucci, Noventa Padovana (PD) (IT); Giovanni Valenti, Bologna (IT); Enrico Rampazzo, Bologna (IT); Massimo Marcaccio, Fermo (IT); Damiano Genovese, Bologna (IT)

(21) Appl. No.: 18/025,408

(22) PCT Filed: Sep. 9, 2021
(86) PCT No.: PCT/IB2021/058199

§ 371 Date: Mar. 9, 2023

(30) Foreign Application Priority Data
Sep. 9, 2020 (IT) 102020000021358

(51) Int. Cl. C09K 11/06; G01N 33/543; B82Y 15/00; B82Y 40/00; B82Y 5/00

(52) U.S. Cl. CPC C09K 11/06; G01N 33/54346; B82Y 15/00; B82Y 40/00; B82Y 5/00; G01N 2474/00

(57) ABSTRACT

It is described a silica nanoparticle comprising: a) a silicate network core with a diameter in a range from 10 nm to 500 nm, preferably from 20 nm to 250 nm, wherein at least a luminophore (dye) is confined in the said silicate network core, or covalently linked to the silicate network of the core because said luminophore (dye) has an anchoring moiety able to form at least a covalent link with the silicate network core, b) a shell layer over the silicate network core a) incorporating dye/s, said shell layer b) with a thickness from 0.5 to 7 nm, preferably from 0.5 to 6 nm, more preferably from 1 to 5 nm, comprising colloidal stabilizer agent/s, selected from the group comprising: sterical or electrostatic antifouling agents or polyether antifouling agents.

### TECHNICAL FIELD OF THE INVENTION

The present invention deals with a new family of Silica nanoparticle/s (SNP/s or NP/s) or Dye Doped Silica Nanoparticles (DDSNPs), showing distinctive improvements when involved in electrochemiluminescent-based analysis.

### STATE OF THE ART

The quantification of diagnostic markers, or biomarkers, has an enormous impact in the early diagnosis, from a the research and clinical point of view. In this context, electrochemiluminescence (ECL) appears to be a leading transduction technique for the detection of very low amounts of these molecules.

ECL is based on the electrochemical generation of species that undergo high-energy electron transfer reactions to form light-emitting excited states.

The combination of electrochemical and spectroscopic methods makes ECL a powerful analytical technique, whose main advantageous feature is the remarkable signal-to-noise ratio due to the absence of a light source for excited states generation.

ECL boasts also excellent spatial and temporal control with the possibility of performing rapid measurements on small sample volumes.

Thanks to these features, ECL has been used for immunoassay and ultrasensitive detection of a wide range of analytes in different fields like medical diagnostics, environmental analysis, and (bio)sensors fabrication.

The subsequent application of ECL principles in microscopy allowed new frontiers and new applications in particular for multiplexing analysis, making the investigation of the ECL mechanisms possible at nanoscale level, especially for sensor application and for biological characterizations.

For these reasons, ECL microscopy is a very promising technique for the surface-confined mapping and quantification of several extremely diluted analytes.

The most used strategy to generate ECL in an aqueous environment is based on the so-called oxidative-reduction co-reactant mechanism where tri-n-propylamine (TPrA) is used as sacrificial co-reactant and tris(2,2′-bipyridine)ruthenium(II) ([Ru(bpy)₃]²⁺) as luminophore, allowing a tremendous number of applications.

This strategy is employed also in commercialized ECL-based immunoassays developed by Roche Diagnostics (Elecsys®) and Meso Scale Diagnostics.

Chasing an ever-increasing sensitivity, many researchers tried to combine ECL with nanomaterials, such as nanoparticles, using them as dyes or co-reactants.

In particular, dye-doped silica nanoparticles (DDSNPs) have proved to be a very interesting option as ECL dyes, due to their several advantages such as (i) an enhanced signal intensity (up to a potential thousand-fold increase) thanks to a large number of inner active dyes, (ii) a simple and versatile synthetic schemes for their preparation, which typically afford high colloidal stability in water, and (iii) an easy bioconjugation.

A potential problem related to the use of DDSNPs is that the process leading to the formation of the emitting excited state in ECL is much more complex than in photo-luminescence.

Particularly in the case of DDSNPs, the generation of the ECL signal typically starts from the oxidation of the coreactant (whose choice is thus of particular importance) at the electrode surface; the oxidized species and their products, which are radical species with a limited lifetime, has to diffuse to quickly reach the ECL probes inside the silica matrix, finally generating the emitting excited state.

This implies that to increase the sensitivity offered by DDSNPs, their dimension should be optimized considering that the amount of ECL-active species is strictly connected to the NP volume.

At the same time, in excessively large particles, this advantage is counterbalanced by the fact that too many probes are not reachable by the diffusing radical species.

Moreover, the nature of NP surface, including its shell, should be engineered to make the NPs colloidally stable in water and endowed with groups suitable for proper derivatization but at the same time to allow the fast diffusion of the radical; in particular thin shells with an overall negative ζ-potential are expected to give the highest signals. Finally, porosity is also expected to play an important and favourable role, thus the synthetic procedure should consider this effect.

Therefore, there is the need to develop new particle/s that allow/s to improve the analytical signal, in particular when involved in electrochemiluminescent-based analysis.

### SUMMARY OF THE INVENTION

The present invention deals with new a new Silica nanoparticle or Dye Doped Silica Nanoparticle (DDSNP) comprising:
a) a silicate network core with a diameter in a range from 10 nm to 500 nm, preferably from 20 nm to 250 nm, wherein at least a luminophore (dye) is confined in the said silicate network core, or covalently linked to the silicate network of the core because said luminophore (dye) has an anchoring moiety able to form at least a covalent link with the silicate network core, wherein this anchoring moiety is preferably a hydrophobic anchoring moiety, more preferably an alkoxysilane moiety, even more preferably a trialkoxysilane moiety;
b) a shell layer over the doped silicate network core a) incorporating dye/s, said shell layer b) with a thickness from 0.5 to 7 nm, preferably from 0.5 to 6 nm, more preferably from 1 to 5 nm, comprising a colloidal stabilizer agent/s, such as sterical or electrostatic antifouling agent or polyether antifouling agents, preferably a stabilizer agent comprising at least a chain structure (cs) having an anchoring moiety cs-1) onward/projecting towards the silicate network core a) incorporating dye/s and a hydrophilic moiety cs-2) outward/projecting outwards from the silicate network core a) incorporating dye/s, preferably the anchoring moiety cs-1) is a hydrophobic anchoring moiety, more preferably an alkoxysilane moiety, even more preferably a trialkoxysilane moiety and/or the hydrophilic moiety cs-2) comprises a polyether moiety, more preferably a -PEGn-OH [-Poly(ethylene glycol)n-OH] moiety wherein n is from 3 to 100, preferably from 4 to 50, more preferably 4, 5, 6, 7, 8, 9 or 10;
wherein the ζ potential of the external surface of the silica nanoparticle is neutral or negative.

It is a further object of the present invention the above mentioned silica nanoparticle/s wherein the shell layer b) comprises a bio-linker (bl) and/or a chemical-linker (cl) and/or a biochemical-linker (bcl) as described below. In the present invention.

It is a further object of the present invention a process for manufacturing a silica nanoparticle/s according to the present invention, said process comprising the Reverse Micro-Emulsion (RME) method wherein the following steps are present:
i) Preparing a water-in-oil emulsion stabilized with non-ionic surfactant/s, and eventually by the presence of co-surfactant/s,
ii) Adding to the water portion of the stabilized water-in-oil emulsion obtained in step i) a luminophore having an anchoring moiety, a silica precursor, and a base to form the dye-doped silicate network core a) of the nanoparticle,
iii) Coating the silicate network core a) incorporating dye/s obtained in step ii) by adding a colloidal stabilizer agent, to obtain a shell layer b) of the nanoparticle.

As a further embodiment of the process according to the present invention, in step iii) it is also added a linker selected from the group comprising: bio-linker and/or biochemical-linker and/or chemical-linker, as defined according to the present invention, and/or, said process comprises the step:
iv) Purifying the nanoparticle obtained in step iii) from non-ionic surfactant and oil.

Given the present invention, the combination of highly sensitive techniques such as ECL with nanotechnology sparkled new analytical applications in particular for immunoassay-based detection systems. In this context, silica nanomaterials according to the present invention, in particular, dye-doped silica nanoparticles (DDSNPs) according to the present invention, are of particular interest, since they can offer several advantages in terms of sensitivity and performance.

According to the present invention, as a preferred embodiment of the silica nanoparticle/s or dye-doped silica nanoparticle/s (DDSNP/s), have been synthesized two sets of monodispersed and biotinylated [Ru(bpy)₃]²⁺-doped silica nanoparticles, named bio-Triton@RuNP and bio-Igepal@RuNP, respectively.

They were obtained following the process according to the present invention, said process comprising the reverse microemulsion method, using two different types of non-ionic surfactants.

Controlling the synthetic procedures according to the present invention, it is possible to obtain silica nanoparticle/s (SNP/s or NPs) or Dye-Doped Silica Nanoparticle/s (DDSNP/s) according to the present invention, offering highly intense signal, using tri-n-propylamine (TPrA) as co-reactant, in particular with bio-Triton@RuNps being more efficient than bio-Igepal@RuNP.

Interestingly, although only a small portion of the around 4800 complexes, i.e. luminophore (dye) according to the present invention, contained in each silica NP was involved in signal generation, when used in ECL analytical mode, the silica nanoparticles or Dye-Doped Silica Nanoparticle/s (DDSNP/s) according to the present invention: BioTriton@RuNPs reveal ECL intensity 8.5-fold higher compared to a system mimicking a commercial ECL-based immunoassay system.

In addition, the silica NPs or Dye-Doped Silica Nanoparticle/s (DDSNP/s) according to the present invention showed an improved ECL stability because of the silica matrix, increasing even more their potential performance.

It is a further object of the present invention the use of the silica nanoparticle/s (NPs) or Dye-Doped Silica Nanoparticle/s (DDSNP/s) according to the present invention, in particular as a probe, in ECL microscopy techniques and suggests a possible further signal increase achievable acting on the synthetic procedure, opening new promising paths towards more sensitive in ECL-based analysis, in particular ECL-based immunoassay, with applications for biosensing and point-of-care devices.

### LIST OF FIGURES

Particular embodiments of the invention are described in detail herein below, as a way of example and not limited to, with reference to the attached figures, wherein:

FIG. 1. shows a schematic representation of the Reverse Micro Emulsion synthesis of the Dye Doped Silica Nanoparticles (DDSNPs), such as [Ru(bpy)₃]²⁺ doped silica nanoparticles, identified as bio-Triton@RuNP and bio-Igepal@RuNP, respectively, according to the present invention.

FIG. 2. shows: (A) TEM Images of Silica NPs top: bio-Igepal@RuNP, bottom: bio-Triton@RuNP. (scale bar 200 nm); (B) Silica core diameters distributions computed by TEM. left: bio-Igepal@RuNP and right bio-Triton@RuNP; (C-D) Normalized absorption and phosphorescence quantum yield of bio-Igepal@RuNP (black continuous line), bio-Triton@RuNP (grey continuous line) and [Ru(bpy)₃]²⁺ (dashed line) in water as reference for comparison.

FIG. 3. shows: (A) ECL intensity potential curves in the presence of TPrA 180 mM in a 1 nM solution of bio-Igepal@RuNp (continuous line) and of bio-Triton@RuNp (dashed line).

Cyclic voltammetries with voltage scanned between 0 V and +1.6 V, scan rate 0.1 V s⁻¹. Glassy Carbon electrode referred to Ag/AgCl. Pt spiral as counter electrode. PMT bias 750V. (B) The heterogeneous mechanism for the “oxidative-reduction” co-reactant ECL generation obtained using 2.8 μm beads labelled with bio-Triton@RuNp (ys) through a streptavidin (gt)-biotin (rt) bond.

Tri-n-propylamine (TPrA) is oxidized at the electrode, generating the radical cation (TPrA^{+•}), which deprotonates, forming the radical (TPrA′). The radical and radical cation reacts with the ECL luminophore [Ru(bpy)₃]2+(yd), inside the bio-Triton@RuNp located on magnetic beads (rss). (C) ECL imaging of 2.8 μm single bead labelled with biotinylated [Ru(bpy)₃]²⁺ complex (beads@bio-Ru) and (D) with bio-Triton@RuNp (beads@Triton). They were obtained by applying a constant potential of 1.4 V (vs. Ag/AgCl) for 4 s in 180 mM TPrA and 0.2 M phosphate buffer (PB). Pt wire as counter electrode. EMCCD camera coupled with a potentiostat. Integration time, 8 s; magnification, ×100; Scale bar, 5 μm. (E) Comparison of the beads profile lines (black line, Beads@bio-Ru; grey line, Beads@Triton). Inset of the comparison between integrated intensity values calculated for Beads@bio-Ru (black) and Beads@Triton (grey), error bar shows the standard deviation (n=9).

FIG. 4. shows the Hydrodynamic diameter distribution with undersize curve (first row) and TEM images (second row) for bio-Triton@RuNP (A) and bio-Igepal@RuNP (B).

FIG. 5. shows: A) Phosphorescence lifetime decays (circle) and fitting (line) of bio-Triton@RuNP (grey, τₘ=790 ns) and bio-Igepal@RuNP (black, τₘ=618 ns); B) Phosphorescence lifetime fitting residuals of bio-Triton@RuNP (left) and bio-Igepal@RuNP (right).

FIG. 6. shows: ECL intensity potential curves in the presence of TPrA 180 mM (dashed line) or DBAE 30 mM (continuous line) in a 10 nM solution of bio-Triton@RuNp. Cyclic voltammetries with voltage scanned between 0 V and +1.6 V, scan rate 0.1 V s⁻¹. Glassy Carbon electrode referred to Ag/AgCl. Pt spiral as counter electrode. PMT bias 750V.

FIG. 7. shows confocal images of beads@bio-Ru (a) and beads@RuNPs (b), λexc=401 nm, emission filter 595/50 nm. Time-gated FLIM images (t>100 ns) of beads@bio-Ru (c) and beads@RuNPs (d), λexc=405 nm, emission filter longpass 560 nm. The average photon arrival time («fast» lifetime) is represented by the color greyscale.

FIG. 8. shows optical (left column) and respective ECL images (right column) of 2.8 μm single bead labelled with bio-Triton@RuNp (beads@Triton). They were obtained by applying a constant potential of 1.4 V (vs. Ag/AgCl) for 4 s in 180 mM TPrA and 0.2 M phosphate buffer (PB). Pt wire as counter electrode. Integration time: 8 s; magnification: ×100; Scale bar, 5 μm.

FIG. 9. shows optical (left column) and respective ECL images (right column) of 2.8 μm single bead labelled with biotinylated Ru(bpy)₃²⁺ complex (beads@bio-Ru). They were obtained by applying a constant potential of 1.4 V (vs. Ag/AgCl) for 4 s in 180 mM TPrA and 0.2 M phosphate buffer (PB). Pt wire as counter electrode. Integration time: 8 s; magnification: ×100; Scale bar: 5 μm.

FIG. 10. shows optical (left column) and respective ECL images (right column) of 2.8 μm single bead labelled with (A) bio-Triton@RuNp (beads@Triton) and (B) with biotinylated Ru(bpy)₃²⁺ complex (beads@bio-Ru). They were obtained by applying a constant potential of 1.4 V (vs. Ag/AgCl) for 2 s in 180 mM TPrA and 0.2 M phosphate buffer (PB). Pt wire as counter electrode. EMCCD camera coupled with a potentiostat. Integration time: 4 s; magnification: ×40; Scale bar: 5 μm.

FIG. 11. shows ECL intensity stability of 2.8 μm single bead labelled with (A) biotinylated Ru(bpy)₃²⁺ complex (beads@bio-Ru) and (B) with bio-Triton@RuNp (beads@Triton).

ECL images acquired each 200 ms (integration time) and ECL intensity integrated plotted against time.

Notice that ECL intensity of beads@bio-Ru is multiplied for a factor 4 for easy comparison with ECL intensity of beads@Triton.

ECL intensities have a drop of 74% and 40% for beads@bio-Ru and beads@Triton respectively.

They were obtained by applying a constant potential of 1.4 V (vs. Ag/AgCl) for 4 s in 180 mM TPrA and 0.2 M phosphate buffer (PB). Pt wire as counter electrode.

### DETAILED DESCRIPTION OF THE INVENTION

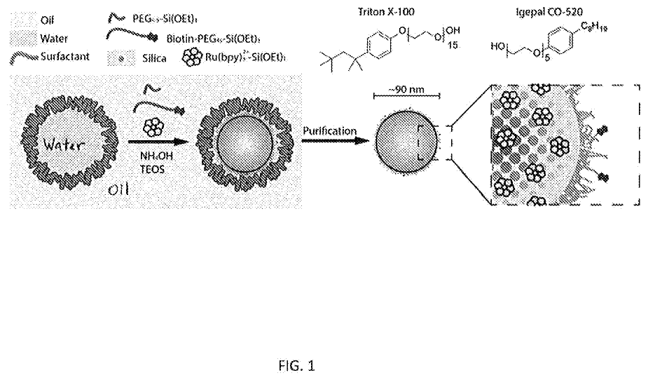

The applicant surprisingly and unexpectedly developed a new Silica nanoparticle or Dye Doped Silica Nanoparticle (DDSNP) (e.g. see FIG. 1) comprising:
a) a silicate network core with a diameter in a range from 10 nm to 500 nm, preferably from 20 nm to 250 nm, wherein at least a luminophore (dye) is confined in the said silicate network core, or covalently linked to the silicate network of the core because said luminophore (dye) has an anchoring moiety able to form at least a covalent link with the silicate network core, wherein this anchoring moiety is preferably a hydrophobic anchoring moiety, more preferably an alkoxysilane moiety, even more preferably a trialkoxysilane moiety (e.g. see FIG. 1);
b) a shell layer over/sheath incorporating the doped silicate network core a) incorporating dye/s, said shell layer b) with a thickness from 0.5 to 7 nm, preferably from 0.5 to 6 nm, more preferably from 1 to 5 nm, comprising a colloidal stabilizer agent/s, such as sterical or electrostatic antifouling agent or polyether antifouling agents, preferably a stabilizer agent comprising at least a chain structure (cs) having an anchoring moiety cs-1) onward/projecting towards the silicate network core a) incorporating dye/s and a hydrophilic moiety cs-2) outward/projecting outwards from the silicate network core a) incorporating dye/s, preferably the anchoring moiety cs-1) is a hydrophobic anchoring moiety, more preferably an alkoxysilane moiety, even more preferably a trialkoxysilane moiety and/or the hydrophilic moiety cs-2) comprises a polyether moiety, more preferably a -PEGn-OH [-Poly(ethylene glycol)n-OH] moiety wherein n is form 3 to 100, preferably from 4 to 50, more preferably 4, 5, 6, 7, 8, 9 or 10 (e.g. see FIG. 1);
wherein the ζ potential of the external surface of the silica nanoparticle is neutral or negative.

It is a further object of the present invention a:

Silica nanoparticle or Dye Doped Silica Nanoparticle (DDSNP) (e.g. see FIG. 1) comprising:
a) a silicate network core with a diameter in a range from 10 nm to 500 nm, preferably from 20 nm to 250 nm, wherein at least a luminophore (dye) is confined in the said silicate network core, or covalently linked to the silicate network of the core because said luminophore (dye) has an anchoring moiety able to form at least a covalent link with the silicate network core, wherein this anchoring moiety is preferably, a hydrophobic anchoring moiety, more preferably an alkoxysilane moiety, even more preferably a trialkoxysilane moiety (e.g. see FIG. 1);
b) a shell layer over/sheath incorporating the doped silicate network core a) incorporating dye/s, said shell layer b) with a thickness from 0.5 to 7 nm, preferably from 0.5 to 6 nm, more preferably from 1 to 5 nm, (e.g. see FIG. 1) comprising:
b1) colloidal stabilizer agent/s, such as sterical or electrostatic antifouling agents or polyether antifouling agents, preferably a stabilizer agent comprising at least a chain structure (cs) having an anchoring moiety cs-1) onward/projecting towards the silicate network core a) incorporating dye/s and a hydrophilic moiety cs-2) outward/projecting outwards from the silicate network core a) incorporating dye/s, preferably the anchoring moiety cs-1) is a hydrophobic anchoring moiety, more preferably an alkoxysilane moiety, even more preferably a trialkoxysilane moiety and/or the hydrophilic moiety cs-2) comprises a polyether moiety, more preferably a -PEGn-OH [-Poly(ethylene glycol)n-OH] moiety wherein n is from 3 to 100, preferably from 4 to 50, more preferably 4, 5, 6, 7, 8, 9 or 10 (e.g. see FIG. 1);
b2) a bio-linker (bl) and/or a chemical-linker (cl) and/or a biochemical-linker (bel) (e.g. see FIG. 1), each of them comprising an anchoring moiety bl-1), cl-1) or bcl-1), respectively, onward/projecting towards the silicate network core a) incorporating dye/s and a hydrophilic moiety bl-2), cl-2) or bcl-2), respectively, outward/projecting outwards from the silicate network core a) incorporating dye/s, wherein:
the terminal part of the hydrophilic moiety bl-2), cl-2) or bcl-2), respectively, comprises at least a biorecognition moiety br-3)), a chemical recognition moiety cr-3)) or biochemical recognition moiety bcr-3)), respectively, in particular, wherein the nature of the “bio-recognition moiety”, “biochemical recognition moiety” or “chemical recognition moiety” depends by the bio-nature, or biochemical nature or chemical nature of the corresponding bio analyte, or biochemical analyte or chemical analyte to be investigated (e.g. see FIG. 1), or
the terminal part of the hydrophilic moiety bl-2), cl-2) or bcl-2), respectively, comprises at least one functional group, useful for the introduction of a biorecognition moiety br-3)), a chemical recognition moiety cr-3)) or biochemical recognition moiety bcr-3)), respectively, said functional group preferably selected from the group comprising amine, —COOH, —N₃, alkyne, alkene, acryloyl, —SH, maleimide, aldehyde, —OH, isothiocyanate, sulfonyl chloride, iodoacetyl, TCT (2,4,6-Trichloro-1,3,5-triazine) or an activated carboxylic group such as NHS and NHS-sulfo esters (N-hydroxysuccinimide and sulfo N-hydroxysuccinimide), TFP ester (2,3,5,6-Tetrafluorophenol), PFP ester (pentafluorophenol), HOBt ester (1-hydroxybenzotriazole), N-acylimidazole, in particular wherein the nature of the “bio-recognition moiety”, “biochemical recognition moiety” or “chemical recognition moiety” depends by the bio-nature, or biochemical nature or chemical nature of the corresponding bio analyte, or biochemical analyte or chemical analyte to be investigated (e.g. see FIG. 1).
   wherein the ζ potential of the external surface of the silica nanoparticle is neutral or negative.

The Silicate Network Core

The silicate network core is derived by a silica precursor or a mixture of silica precursors selected from the group comprising alkoxysilane precursors such as TEOS (tetraethoxysilane), TMOS (tetramethoxysilane), tetrabuthoxysilane, 1,2-Bis(triethoxysilyl)ethane, 1,2-Bis(trimethoxysilyl)ethane, organoalkoxysilane 1-4 such as: 1,1′-(ethane-1,2-diyl)bis(3-(3-(triethoxysilyl)propyl)u rea), 1,1′-(hexane-1,6-diyl)bis(3-(3-(triethoxysilyl)propyl)u rea), 1,1′-(1,4-phenylene)bis(3-(3-(triethoxysilyl)propyl)urea), 1,1′-(ethane-1,2-diyl)bis(1-phenyl-3-(3-(triethoxysilyl)propyl)urea).

The formation of the silicate network core, which is formed from the hydrolysis and condensation processes of organosilicates, leads to the substantially irreversible immobilization of the luminophore (dye) in the core of the silicate network of the silica particle.

According to the present invention, the selected dye is derivatized in order to be covalently linked to the silicate network of the core of said silica nanoparticle avoiding any leaking of the dye, i.e. it is functionalized with anchoring moiety preferably a hydrophobic anchoring moiety, more preferably an alkoxysilane moiety, even more preferably a trialkoxysilane moiety, in order to be confined in the silicate network core of said silica nanoparticle.

The Luminophore (Dye)

The luminophore (dye) according to the present invention is a luminophore (dye) having a functionality useful for the introduction of an anchoring moiety, preferably hydrophobic anchoring moiety, more preferably an alkoxysilane moiety, even more preferably a trialkoxysilane moiety, for the linking of the luminophore (dye) to the silicate network core, said functionality preferably selected from the group comprising: amine, —COOH, —N₃, alkyne, alkene, acryloyl, —SH, maleimide, aldehyde, —OH, isothiocyanate, sulfonyl chloride, iodoacetyl, TCT (2,4,6-Trichloro-1,3,5-triazine) or an activated carboxylic group such as NHS and NHS-sulfo esters (N-hydroxysuccinimide and sulfo N-hydroxysuccinimide), TFP ester (2,3,5,6-Tetrafluorophenol), PFP ester (pentafluorophenol), HOBt ester (1-hydroxybenzotriazole), N-acylimidazole.

Preferably, the luminophore (dye) is a metal complex, more preferably selected from the group comprising:
Ruthenium(II) polypyridine derivatives, such as Ru(bpy)₃²⁺ where bpy is tris(2,2′-bipyridine), Ru(phen)₃²⁺ where phen is 1,10-Phenanthroline, Ru(bpy)₂(bps), Ru(phen)₂ (bps)—where bps is 4,7-diphenyl-1,10-phenanthroline disulfonate, and derivates thereof with general structure [Ru(bpy)₃-n (bps)n]²⁻²ⁿ or [Ru(phen)₃₋ₙ (bps)ₙ]²⁻²ⁿ wherein n is 1, 2 or 3, Ru(phen)₂ (dppz)²⁺ or Ru(bpy)₂ (dppz)²⁺—where dppz is dipyrido[3,2-a:2,3-c]phenazine.
Cyclometalated Ir(III) metal complexes homoleptic, such us Ir(C\hat{ }N)₃ where CAN is a monoanionic ligand such as 2-Phenylpyridine, and heteroleptic Ir(C\hat{ }N)₂ (L\hat{ }L) where CAN is a monoanionic ligand such as 2-Phenylpyridine and LAL is 2,2′-bipyridine
   or
   the luminophore (dye) is an organic luminophore, more preferably selected from the following derivatives: anthracene derivatives, xanthene dyes derivatives, cyanine derivatives, bodipy dye derivatives and coumarin dye derivatives.

The ζ Potential

The ζ potential is a well-known physic parameter, which is defined as the electrical potential at the Hydrodynamic Plane of Shear.

The Bio-Linker/Biochemical-Linker/Chemical-Linker

The bio-linker (bl) and/or biochemical-linker (bcl) and/or chemical-linker (cl) according to the present invention is/are chosen according to the nature of the analyte to be investigated, i.e. according to the bio-nature, or biochemical nature or chemical nature of the corresponding bio analyte, or biochemical analyte or chemical analyte to be investigated.

Preferably, the bio-linker (bl) and/or a chemical-linker (cl) and/or a biochemical-linker (bcl) comprised in the shell layer b) according to the present invention, comprises an anchoring moiety bl-1), cl-1) or bcl-1), respectively, onward/projecting towards the silicate network core a) incorporating dye/s and a hydrophilic moiety bl-2), cl-2) or bcl-2), respectively, outward/projecting outwards from the silicate network core a) incorporating dye/s, wherein:
the terminal part of the hydrophilic moiety bl-2), cl-2) or bcl-2), respectively, comprises at least a biorecognition moiety br-3)), a chemical recognition moiety cr-3)) or biochemical recognition moiety bcr-3)), respectively, in particular, wherein the nature of the “bio-recognition moiety”, “biochemical recognition moiety” or “chemical recognition moiety” depends by the bio-nature, or biochemical nature or chemical nature of the corresponding bio analyte, or biochemical analyte or chemical analyte to be investigated, or
the terminal part of the hydrophilic moiety bl-2), cl-2) or bcl-2), respectively, comprises at least one functional group, useful for the introduction of a biorecognition moiety br-3)), a chemical recognition moiety cr-3)) or biochemical recognition moiety bcr-3)), respectively, said functional group preferably selected from the group comprising amine, —COOH, —N₃, alkyne, alkene, acryloyl, —SH, maleimide, aldehyde, —OH, isothiocyanate, sulfonyl chloride, iodoacetyl, TCT (2,4,6-Trichloro-1,3,5-triazine) or an activated carboxylic group such as NHS and NHS-sulfo esters (N-hydroxysuccinimide and sulfo N-hydroxysuccinimide), TFP ester (2,3,5,6-Tetrafluorophenol), PFP ester (pentafluorophenol), HOBt ester (1-hydroxybenzotriazole), N-acylimidazole, in particular wherein the nature of the “bio recognition moiety”, “biochemical recognition moiety” or “chemical recognition moiety” depends by the bio-nature, or biochemical nature or chemical nature of the corresponding bio analyte, or biochemical analyte or chemical analyte to be investigated,

More preferably, the bio-linker (bl) and/or a chemical-linker (cl) and/or a biochemical-linker (bcl) comprised in the shell layer b) according to the present invention, comprises an anchoring moiety bl-1), cl-1) or bcl-1), respectively, onward/projecting towards the silicate network core a) incorporating dye/s, said anchoring moieties being a hydrophobic anchoring moiety, even more preferably an alkoxysilane moiety, the most preferred a trialkoxysilane moiety and/or a hydrophilic moiety bl-2), cl-2) or bcl-2), respectively, outward/projecting outwards from the silicate network core a) incorporating dye/s, said hydrophilic moieties comprising a polyether moiety, even more preferably a -PEGn-OH [-Poly(ethylene glycol)n-OH] moiety wherein n is from 3 to 100, preferably from 4 to 50, while:
the terminal part of the hydrophilic moiety bl-2), cl-2) or bcl-2), respectively, comprises at least a biorecognition moiety br-3)), a chemical recognition moiety cr-3)) or biochemical recognition moiety bcr-3)), respectively, in particular, wherein the nature of the “bio-recognition moiety”, “biochemical recognition moiety” or “chemical recognition moiety” depends by the bio-nature, or biochemical nature or chemical nature of the corresponding bio analyte, or biochemical analyte or chemical analyte to be investigated, or
the terminal part of the hydrophilic moiety bl-2), cl-2) or bcl-2), respectively, comprises at least one fictional group, useful for the introduction of a bio recognition moiety br-3)), a chemical recognition moiety cr-3)) or biochemical recognition moiety bcr-3)), respectively, said functional group preferably selected from the group comprising amine, —COOH, —N₃, alkyne, alkene, acryloyl, —SH, maleimide, aldehyde, —OH, isothiocyanate, sulfonyl chloride, iodoacetyl, TCT (2,4,6-Trichloro-1,3,5-triazine) or an activated carboxylic group such as NHS and NHS-sulfo esters (N-hydroxysuccinimide and sulfo N-hydroxysuccinimide), TFP ester (2,3,5,6-Tetrafluorophenol), PFP ester (pentafluorophenol), HOBt ester (1-hydroxybenzotriazole), N-acylimidazole, in particular wherein the nature of the “bio recognition moiety”, “biochemical recognition moiety” or “chemical recognition moiety” depends by the bio-nature, or biochemical nature or chemical nature of the corresponding bio analyte, or biochemical analyte or chemical analyte to be investigated.

It is a further object of the present invention the use of the silica nanoparticle/s or Dye-Doped Silica Nanoparticle/s (DDSNP/s) according to the present invention as new diagnostic tools and in particular the silica nanoparticle/s (SNP/s or NP/s) or Dye-Doped Silica Nanoparticle/s (DDSNP/s) according to the present invention to be used for, or the use thereof in applications for the detection, labelling and imaging of bio-molecules and/or biochemical molecules and or chemical molecules.

Another object of the present disclosure is the use of the above silica nanoparticle in therapy and diagnostics.

A particularly preferred disclosure of the silica nanoparticle of the present invention is a probe, according to the definitions as commonly intended in this technical field and also according to the definitions provided in the above mentioned WO2010013136 and WO2010013137.

Another object of the present invention is the use of the above silica nanoparticle in analytical chemistry, in particular as a probe as commonly intended in this technical field.

Another object of the present invention is a diagnostic composition comprising a suitable amount of the above silica nanoparticle.

Process

An example of the process of manufacture of the silica nanoparticle/s or Dye Doped Silica Nanoparticle/s (DDSNP/s) according to the present invention, said process comprising the Reverse Micro-Emulsion (RME) method, is schematically represented in FIG. 1 wherein the process of the silica nanoparticle/s or Dye Doped Silica Nanoparticle/s (DDSNP/s) according to the present invention, starts with the preparation of the water-in-oil emulsion stabilized with non-ionic surfactants, such as polyethylene glycol tert-octylphenyl ether (Triton® X-100) or polyoxyethylene (12) iso-octylphenyl ether (Igepal® CO-520), eventually, if necessary, in presence of co-sufactant, such as 1-hexanol, then upon the addition of: a luminophore (dye) or a luminophore (dye) having an anchoring moiety, such as [Ru(bpy)₃]²⁺ triethoxy silane derivative (Ru(bpy)₃²⁺—Si(OEt)₃), a silica precursor, such as TEOS and a base, such as ammonium hydroxide (NH₄OH), the silicate network core a) incorporating dye/s of the silica nanoparticle/s or Dye Doped Silica Nanoparticle/s (DDSNP/s) according to the present invention, is formed.

The silicate network cores a) incorporating dye/s so obtained are then coated with a shell layer b) comprising a stabiliser agent such as antifouling agent like polyethylenglycol triethoxy silane derivative (PEG₆₋₉Si(OEt)₃) and with bio linkers such as biotin tagged polyethylenglycol triethoxysilane derivative (Biotin-PEG₄₅-Si(OEt)₃). The synthesis ends with the purification of the silica nanoparticle/s or Dye Doped Silica Nanoparticle/s (DDSNP/s) according to the present invention, from the surfactant and the oil phase.

As a non-ionic surfactant or co-surfactant according to the present invention is meant: as a non-ionic surfactant is meant a molecule carrying no charge, and presenting a hydrophilic chain, preferably comprising or composed by a polyoxoether, and a hydrophobic tail, preferably said hydrophobic tail selected from the group comprising an alkyl, fluoroalkyl, or steroidal tail; or any non-ionic surfactant and co-surfactant which are well known in the art to be used or involved in the Reverse Micro-Emulsion (RME) method, such as polyoxoethylen alchil phenyl ethers, or polyoxoethylene (n) nonylphenylether wherein

π=5, 9, 12, 40, i.e. polyethylene glycol tert-octylphenyl ether (such as Triton™ X-100), polyoxoethylene nonylphenylethers (such as IGEPAL®), polyoxyethylene (12) isooctylphenyl ether (such as IGEPAL® CA-720), Polyethylene glycol sorbitan monolaurate (such as TWEEN® 20), sorbitan esters (such as Span®), polyethylene glycol alkyl ethers (such as Brij) and 1-hexanol.

It is a further object of the present invention a process for manufacturing a silica nanoparticle or Dye Doped Silica Nanoparticle (DDSNP) according to the present invention, said process comprising the Reverse Micro-Emulsion (RME) (water-in oil/organic phase/hydrocarbon) method wherein the following steps are present:
i) Preparing a water-in-oil emulsion stabilized with non-ionic surfactant/s, and eventually, if necessary, by the presence of co-surfactant/s
ii) Adding to the water portion of the stabilized water-in-oil emulsion obtained in step i) a luminophore (dye) having an anchoring moiety, a silica precursor and a base to form the silicate network core a) incorporating dye/s of the silica nanoparticle or Dye Doped Silica Nanoparticle (DDSNP),
iii) Coating the silicate network core a) incorporating dye/s of the silica nanoparticle or Dye Doped Silica Nanoparticle (DDSNP) obtained in step H) by adding a stabilizer agent to obtain a shell layer b) of the silica nanoparticle or Dye Doped Silica Nanoparticle (DDSNP).

As a preferred embodiment of the process for manufacturing a silica nanoparticle or Dye Doped Silica Nanoparticle (DDSNP) according to the present invention, said process comprises the Reverse Micro-Emulsion (RME) method wherein the following steps are present:
i) Preparing a water-in-oil emulsion stabilized with non-ionic surfactant/s, and eventually, if necessary, by the presence of co-surfactant/s,
ii) Adding to the water portion of the stabilized water-in-oil emulsion obtained in step i) a luminophore (dye) having an anchoring moiety, a silica precursor and a base to form the silicate network core a) incorporating dye/s of the silica nanoparticle or Dye Doped Silica Nanoparticle (DDSNP),
iii) Coating the silicate network core a) incorporating dye/s of the silica nanoparticle or Dye Doped Silica Nanoparticle (DDSNP) obtained in step H) by adding a stabilizer agent to obtain a shell layer b) of the silica nanoparticle or Dye Doped Silica Nanoparticle (DDSNP),
iv) Purifying the silica nanoparticle or Dye Doped Silica Nanoparticle obtained in step iii) from non-ionic surfactant and oil.

As a more preferred embodiment of the process for manufacturing a silica nanoparticle or Dye Doped Silica Nanoparticle (DDSNP according to the present invention, said process comprises the Reverse Micro-Emulsion (RME) method wherein the following steps are present:
i) Preparing a water-in-oil emulsion stabilized with non-ionic surfactant/s, and eventually, if necessary, by the presence of co-surfactant/s
ii) Adding to the water portion of the stabilized water-in-oil emulsion obtained in step i) a luminophore (dye) having an anchoring moiety, a silica precursor and a base to form the silicate network core a) incorporating dye/s of the silica nanoparticle or Dye Doped Silica Nanoparticle,
iii) Coating the silicate network core a) incorporating dye/s of the silica nanoparticle or Dye Doped Silica Nanoparticle obtained in step H) by adding a stabilizer agent and a linker selected from the group comprising: bio-linker and/or biochemical-linked and/or chemical-linker, to obtain a shell layer b) of the silica nanoparticle or Dye Doped Silica Nanoparticle.

As the most preferred embodiment of the process for manufacturing a silica nanoparticle or Dye Doped Silica Nanoparticle (DDSNP) according to the present invention, said process comprises the Reverse Micro Micro-Emulsion (RME) method wherein the following steps are present:
i) Preparing a water-in-oil emulsion stabilized with non-ionic surfactant/s, and eventually, if necessary, by the presence of co-surfactant/s
ii) Adding to the water portion of the stabilized water-in-oil emulsion obtained in step i) a luminophore (dye) having an anchoring moiety, a silica precursor and a base to form the silicate network core a) incorporating dye/s of the silica nanoparticle or Dye Doped Silica Nanoparticle (DDSNP),
iii) Coating the silicate network core a) incorporating dye/s of the silica nanoparticle or Dye Doped Silica Nanoparticle (DDSNP) obtained in step ii) by adding a stabilizer agent and a linker selected from the group comprising: bio-linker and/or biochemical-linked and/or chemical-linker, to obtain a shell layer b) of the silica nanoparticle or Dye Doped Silica Nanoparticle (DDSNP),
iv) Purifying the silica nanoparticle or Dye Doped Silica Nanoparticle (DDSNP) obtained in step iii) from non-ionic surfactant and oil.

In particular, to further push the signal intensity, new silica nanoparticle/s (NP/s) or Dye-Doped Silica Nanoparticle/s (DDSNP/s) according to the present invention has/have been synthesized with a reverse microemulsion method, that allows obtaining a suspension of monodispersed silica nanoparticle/s NPs with greater flexibility in terms of particle size and surface properties and with different surface functionalization, through an excellent control on the synthetic parameters.

In step iii), when the colloidal stabilizer agent and the linker (bio-linker and/or biochemical-linker and/or chemical-linker) is/are added to the water portion of the stabilized water-in-oil emulsion to form the shell layer b) which coats the silicate network core a) (see for instance FIG. 1 and the examples), the anchoring moieties, such as the trialkoxysilane moieties of the colloidal stabilizer agent and the linker, link to the silicate network core a) already incorporating dye/s, thus forming covalent bonds with the surface of said silicate network core a) (see for instance FIG. 1).

The purification step iv) is performed according to the standard procedures in relation to the Reverse Micro-emulsion method as exemplified in the experimental part of the description: i.e., the nanoparticles are isolated from the microemulsion by adding an organic solvent such as aceton or ethanol or methanol and centrifuged multiple times; then the nanoparticles are washed with ethanol and/or water several times possibly with ultrasound/centrifuge.

As an embodiment of the present invention, the applicant has been obtained (synthetic scheme in FIG. 1) two sets of monodispersed Ru(bpy)₃²⁺-doped silica NPs, namely bio-Triton@RuNP and bio-Igepal@RuNP, using two different types of nonionic surfactants (TritonX-100 and Igepal CO-520), [Ru(bpy)₃]²⁺—Si(OEt)₃ derivative as a dye and biotinylated polyethylene glycol-Si(OEt)₃ as biorecognition unit.

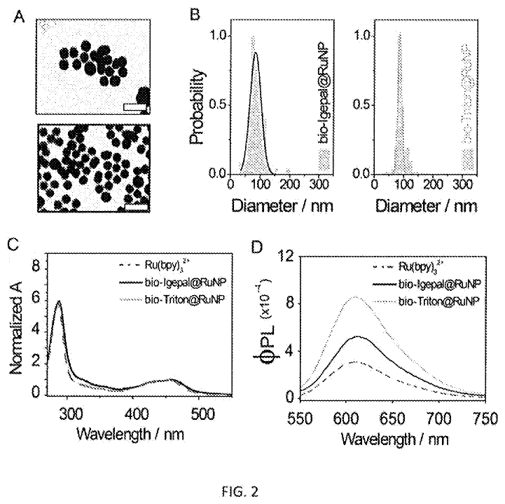

The core (˜90 nm) and hydrodynamic (d_{H}˜150 nm) diameters—determined by TEM and DLS respectively (see FIGS. 2A and 2B)—of bio-Triton@RuNP and bio-Igepal@RuNP are reported in Table 1.

It can be noted that they are almost independent on the surfactant used (see FIG. 4).

TABLE 1
% mol of dyes vs mol of TEOS and number of dyes value of
bio-Triton@RuNP and bio-lgepal@RuNP. Hydrodynamic diameter,
core diameter, polidispersion index (PDI),
ζ-potential measured at pH 6.7.

| | % mol | | d_{H} ± | | | ζ |
|---|---|---|---|---|---|---|
| | dye/mol | no. | SD | d_{H} ± SD | | potential |
| sample | TEOS | dye/NP | [nm] | [nm] | PDI | [mV] |
| bio- | 2 | 4800 | 90 ± 10 | 145 ± 5 | 0.10 | 1.3 ± 0.6 |
| Triton@RuNP | | | | | | |
| bio- | 2 | 3200 | 85 ± 20 | 150 ± 10 | 0.15 | 6.8 ± 1.5 |
| lgepal@RuNP | | | | | | |

The absorption and emission spectra, reported in FIG. 2, show that the peak shape and position of the two sets of NPs are similar to each other, as well as to [Ru(bpy)₃]²⁺ in water solution (dashed line), since silica matrix is not dramatically perturbing the electronic ground state conditions. The phosphorescence quantum yield D and the average lifetime of the dye in bio-Triton@RuNPs (ϕ_{PL}=0.080, τ˜790 ns) show about three-fold increase respect to the free [Ru(bpy)₃]²⁺ dye (ϕ_{PL}=0.028, τ=334 ns) in aerated water solutions, while a lower increase has been observed for bio-Igepal@RuNPs (ϕ_{PL}=0.050, τ˜618 ns)[see FIG. 5]. The increase of the phosphorescence quantum yield and the lengthening of the excited state lifetime can be attributed to the reduced diffusion rate of molecular oxygen in the silica matrix, which depends on the synthetic procedure.

By assuming that the absorption coefficient of the [Ru(bpy)₃]²⁺ complexes is not significantly altered because of their insertion inside the silica matrix, the Applicant was able to estimate (for further details, see Table 1) that each silica nanoparticle (NP or SNP) according to the present invention includes an average of 3200 and 4800 ruthenium complexes for bio-Igepal@RuNP and bio-Triton@RuNP, respectively, despite the same initial doping level (2%). It is noteworthy that, although the number of dyes was higher in bio-Triton@RuNP, in these NPs the ζ-potential remained very close to 0 mV unlike previous results with PluS NPs, while a slightly positive value was found for bio-Igepal@RuNP.

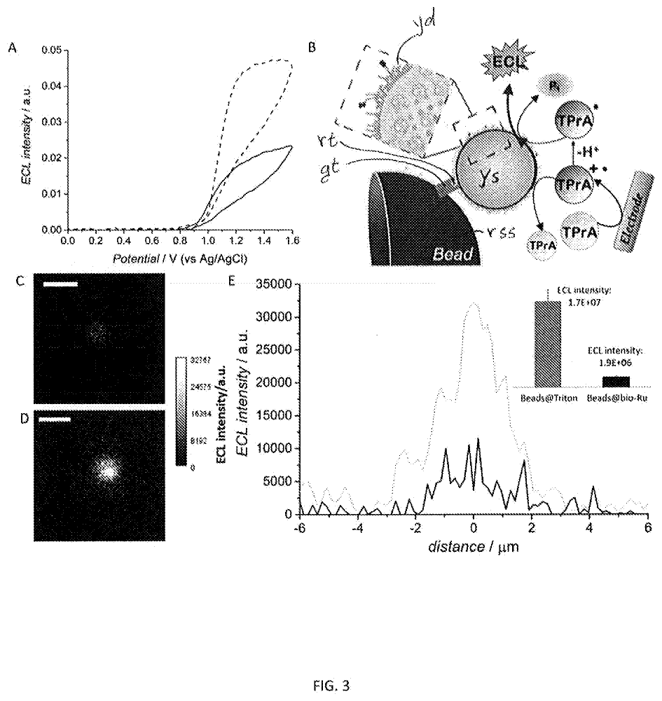

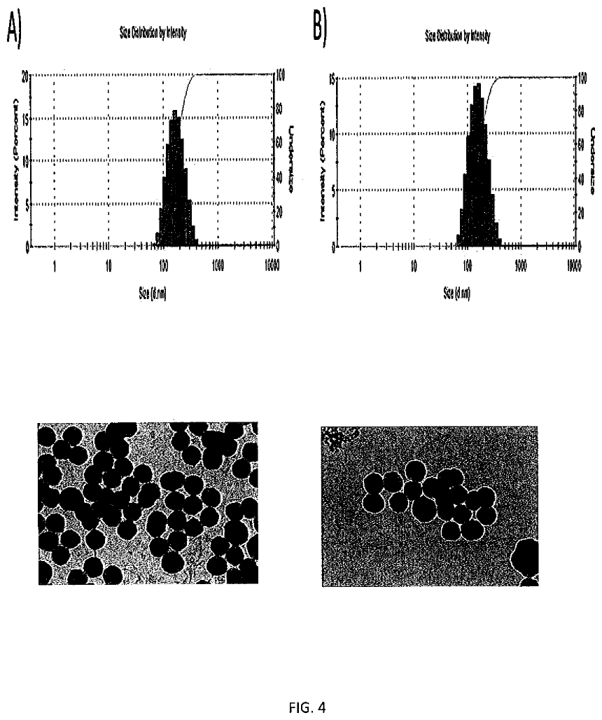

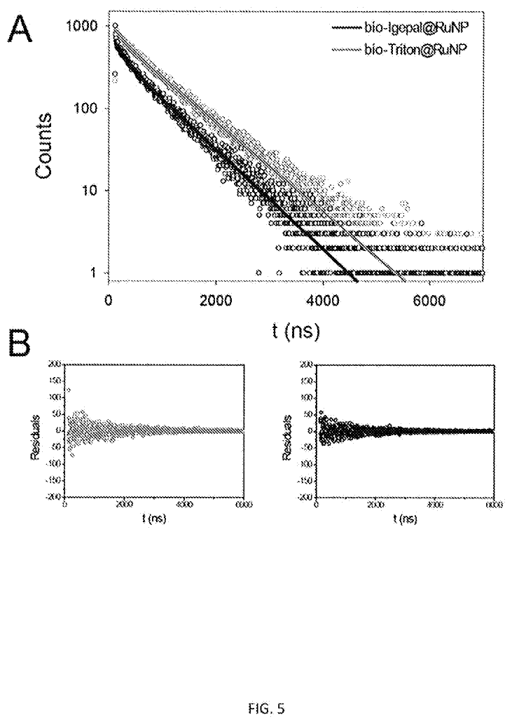

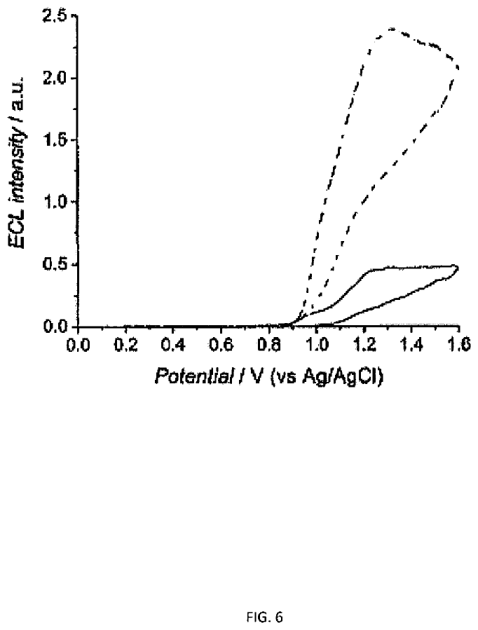

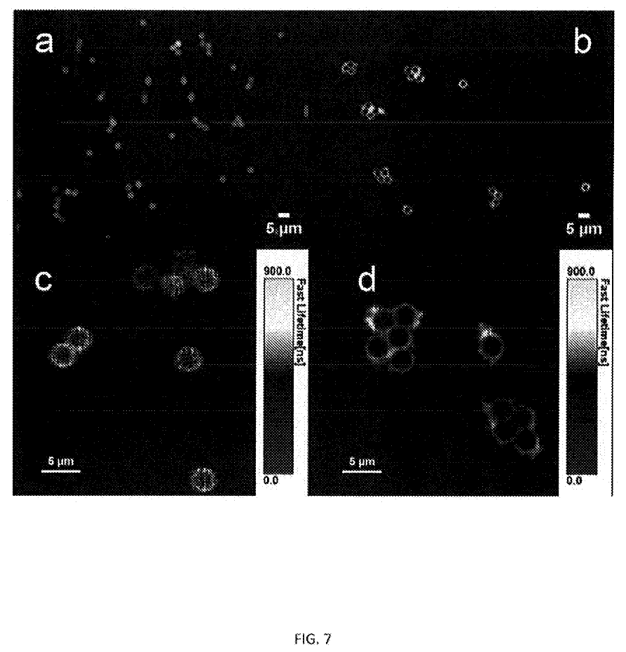

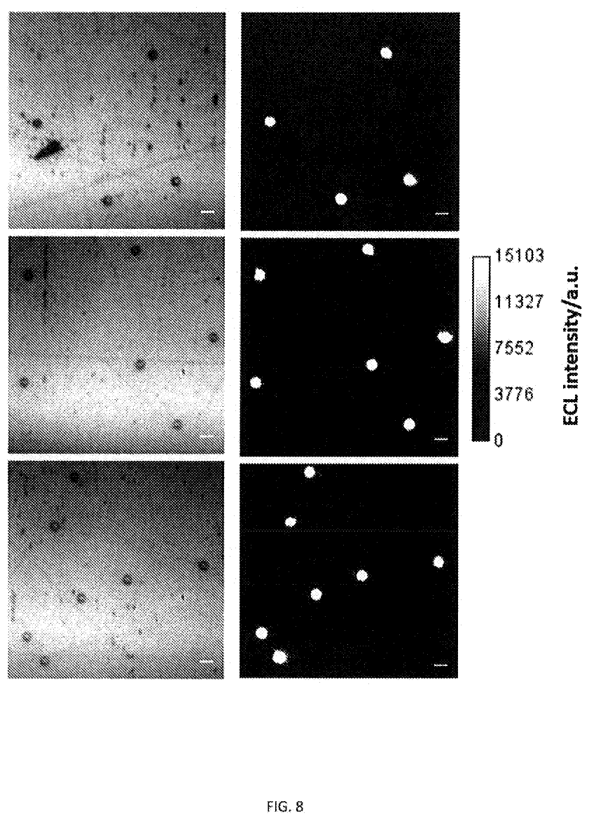

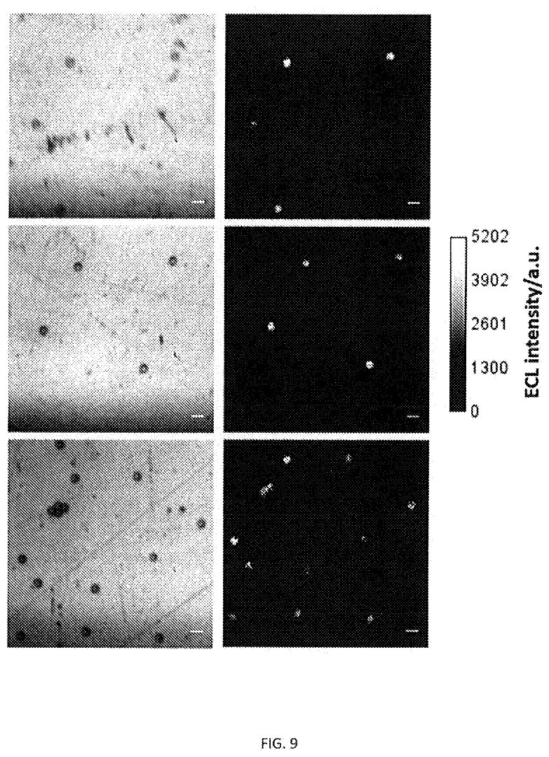

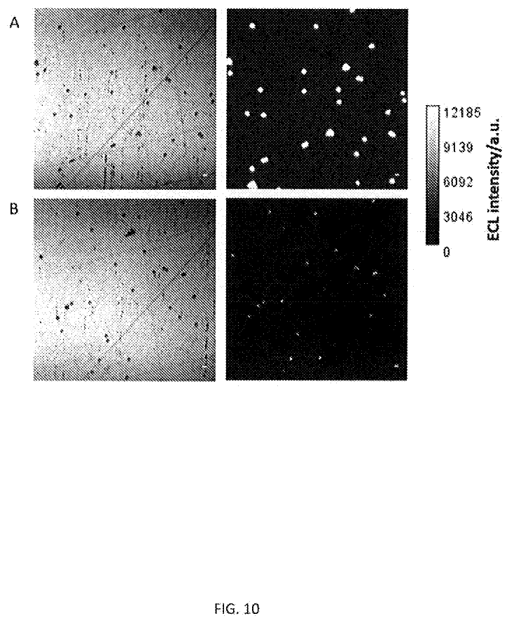

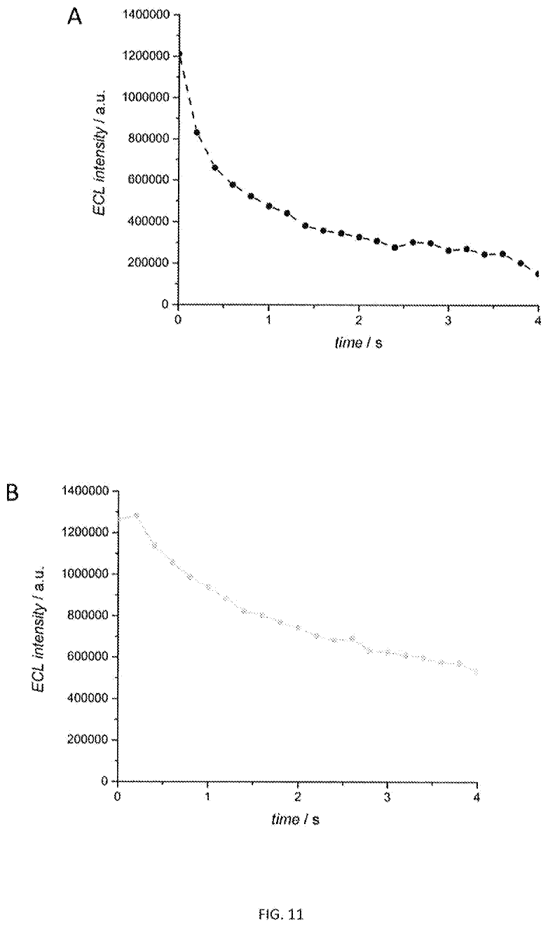

To test the ECL performances of the two sets of NPs, cyclic voltammetry (CV) was performed on 1 nM of NPs using TPrA (180 mM) as sacrificial co-reactant and the ECL intensity was acquired from a photomultiplier tube applying a bias of 750 V. In FIG. 3, the registered ECL intensity was plotted against the potential scanned between 0 V and +1.6V. The general mechanism active in this condition for the ECL generation is based on the so-called oxidative-reduction co-reactant mechanism schematize in FIG. 3A and with the following equation:

TPrA-e⁻↔TPrA^{•+} (1)

TPrA^{•+}↔TPrA^{•}+H⁺ (2)

TPrA^{•}+bio-Triton@RuNps↔P1+bio-Triton@Ru⁻Nps (3)

TPrA^{•+}+bio-Triton@Ru⁻Nps↔TPrA+bio-Triton@Ru*Nps (4)

bio-Triton@Ru*Nps↔bio-Triton@RuNps+hv (5)

where TPrA tri-n-propylamine; bio-Triton@RuNps is the [Ru(bpy)₃]²⁺; bio-Triton@Ru⁻ Nps is the [Ru(bpy)₃]⁺; bio-Triton@Ru*Nps is the [Ru(bpy)₃]²⁺* embedded in the nanoparticle and P1 is the product of the homogeneous TPrA oxidation. It can be clearly seen that bio-Triton@RuNPs show a much higher ECL intensity than bio-Igepal@RuNps. This difference can be only in part explained by the higher doping degree observed for the NPs synthesized with the Triton surfactant.

In fact, even after normalizing the ECL intensity by the number of dyes/NP, bio-Triton@RuNP still displays a higher intensity (see Table 2), thus suggesting that other factors, e.g., the different ζ-potentials and photoluminescence quantum yields, should be considered.

TABLE 2
Maximum ECL intensities obtained in cyclic voltammetry and performed
on bio-Igepal@RuNp and bio-Triton@RuNp (see FIG. 3A)
and ECL intensity normalized for the number of dyes for NP.

| | | | | ECL intensity | Relative ECL |
|---|---|---|---|---|---|
| | ECL intensity | Relative | | normalized for | normalized |
| sample | maximum | ECL intensity | Nodye/NP | Nodye/NP | intensity |
| bio-Triton@RuNP | 0.047 | 2 | 4800 | 9.7E−06 | 1.4 |
| bio-Igepal@RuNP | 0.023 | 1 | 3200 | 7.1E−06 | 1 |

The positive ζ-potential of bio-Igepal@RuNP is, according to previous results, disadvantageous to the ECL emission, most probably because of the electrostatic repulsion between the NP surface and the approaching co-reactant cationic intermediates. For this problem, the ECL generation with NPs so far was restricted to the use of hydrophilic coreactants (such as 2-(dibutylamino)ethanol), thus barring the efficient ECL generation achievable with TPrA.

However, the data show that the Applicant was able with this synthetic strategy to obtain NPs endowed with the correct NPs parameters (e.g. ζ-potentials, biorecognition unit, hydrophobicity, dye distribution and NP size) for an efficient ECL generation using TPrA (see FIG. 4). The enhanced ECL intensity obtained with low concentrations of bio-Triton@RuNPs and the use of TPrA as co-reactant prompted us to test such NPs in ECL imaging, which was unsuccessful with other core-shell NPs likely due to the above detrimental effects. According to the mechanism schematized in FIG. 3B (see also equation 1-5), ECL imaging was performed on single 2.8 μm beads functionalized with either bio-Triton@RuNps (beads@Triton) or a biotinylated antibody labelled with [Ru(bpy)₃]²⁺ complex (beads@bio-Ru), mimicking the analytical approach of commercial ECL-based immunoassay system (FIG. 7).

Interestingly, the ECL intensity obtained in case of beads@Triton was 8.5-fold higher (see FIGS. 3C, D, Table 3), representing a very interesting and promising result.

TABLE 3
ECL intensity at a single bead level of beads@bio-Ru and beads@triton
together with the quantitative parameters of dye present.

| sample | ECL intensity | Ru active site⁻¹ | Lifetime (FLIM) |
|---|---|---|---|
| beads@bio-Ru | 1946131 | 6 | 830 ns |
| beads@Triton | 16588240 | 4800 | 870 ns |

ECL images from 2.8 μm beads functionalized (FIGS. 3C and 3D), from multiple beads (FIGS. 8-10) and respective ECL emission profiles (FIG. 3E), confirm the massive ECL signal enhancement of beads@Triton (grey line) compared to beads@bio-Ru, (black line and see the inset of FIG. 3E).

To quantitively compare the two cases, the amount of Ru immobilised onto the surface of the microbeads was quantified by ICP-MS analysis (Table 4).

TABLE 4
ICP-MS parameters relative to the dye quantification
obtained from the analyses of 500 μL beads@bio-Ru
and beads@Triton having an established area of 4.42E+09 μm².
The concentration of Ruthenium [Ru] is directly obtained from
ICP-MS analysis. Nº Ru is the number of ruthenium
ion obtained bythe product of Ru concentration, the
volume (500 μL) and the Avogadro constant. Ru/μm² is the number
of ruthenium divided by the beads area in 500 μL.

| | | Area of | | | | |
|---|---|---|---|---|---|---|
| | Total | 0.5 mL | | | | |
| sample | area | beads | [Ru]/ppb | [Ru]/M | Nº Ru | Ru/μm² |
| beads@bio- | 7.1E+09 | 4.4E+09 | 0.723 | 7.2E−09 | 2.2E+13 | 4.9E+3 |
| Ru | | | | | | |
| beads@Triton | 7.1E+09 | 4.4E+09 | 479 | 4.7E−06 | 1.4E+16 | 3.2E+6 |

As it can be seen, the dye concentration was about 660 times higher in the case of beads@Triton, a value that is in line with an expected similar occupancy of the active sites onto the microbead surface by either the biotinylated Ru-derivatized antibody or bio-Triton@RuNps, which contain 6 and 4800 [Ru(bpy)₃]²⁺ complexes, respectively. This means that only a small portion of the Ru complexes (around 1.3%, assuming a yes-or-not system) participate in the vast (750%) increase of ECL emission to the commercially available approach.

In the present case, bio-Ru dyes show a lifetime nearly as long as [Ru(bpy)₃]²⁺ in Triton NPs (FLIM images in FIG. 7, Table 3), i.e. noticeably longer than the one of [Ru(bpy)₃]²⁺ complex in water. This means that the increase of luminescence lifetime and quantum yield caused by the inclusion of the complex inside the silica matrix can be only in a minimal part the cause of the observed signal increase.

Interestingly, the ECL from beads@Triton showed improved stability compared with beads@bio-Ru (FIG. 11) thanks to the nano-environment of silica matrix that protects against undesired electrochemical reactions, increasing their potential performance.

This result, already very promising in se, suggests that acting on the synthetic procedure can lead to an even more pronounced signal increase. Further investigation in this direction will be performed for implementing the combination between NPs and the ECL imaging.

To conclude, the Applicant synthesized a new family of silica NPs, according to the present invention such as named bio-Triton@RuNps, obtained following the reverse microemulsion method and derivatized with biotin.

Using this synthetic approach, oppositely to what was observed with another kind of NPs already known in the state of the art, the high dye doping degree (ca 4800 complexes every NP) did not bring to a positive surface charge thus allowing a high ECL emission. Moreover, by using TPrA as coreactant, these DDSNPs lead to a remarkable enhancement of ECL signal compared to the conditions mimicking the commercial ECL-based immunoassay system (i.e., based on an antibody labelled with 6 dyes). The silica matrix can increase the stability of the ECL signal, increasing, even more, the potential performances of these NPs. These results support the use of the silica nanoparticle/s (SNP/s or NP/s) or Dye-Doped Silica Nanoparticle/s (DDSNP/s) according to the present invention, in ECL microscopy techniques and also opens a new promising path towards more sensitive analyte detection, even in biosensing and in point-of-care devices. The improvement of the number of complexes active in the generation of higher ECL signals and an even larger increase in the ECL stability represents a further push to steer possible impactful investigations in this direction.

### EXAMPLES

Methods

Chemicals

Igepal CO-520 (polyoxoethylene nonylphenylether, MW_{avg}=2200 g/mol), Triton X-100 (polyethylene glycol tert-octylphenyl ether, MW_{avg}=5900 g/mol), tetraethyl ortosilicate (TEOS, MW=208.33 g/mol, ≥99.99%), aqueous ammonia solution (NH₄OH, MW=40.08 g/mol, 28-30 wt % in water), O-[2-(Biotinyl-amino)ethyl]-O′-[3-(N-succinimidyloxy)-3-oxopropyl]polyethylene glycol (biotin-PEG-NHS, MW=3000 g/mol), Tri-n-propylamine (TPrA, MW=143.27 g/mol, ≥98% V/V), sodium phosphate monobasic dihydrate (NaH₂PO₄·2H₂O, MW=119.98 g/mol, ≥99%), sodium phosphate dibasic (Na₂HPO₄, MW=141.96 g/mol, ≥99%), phosphoric acid (H₃PO₄, MW=98.00 g/mol, ≥85%), 4,4′-dimethyl-2,2′-bipyridine (MW=184.24 g/mol, 99%), Li-diisopropylamine (MW=107.12 g/mol, ≥97%), 1,3-bromopropane (MW=201.89 g/mol, ≥99.9%), potassium phthalimide (MW=185.22 g/mol, ≥98%), n-hexanol (anhydrous, MW=102.17 g/mol, ≥99%), Cyclohexane (MW=84.16 g/mol, ≥99%), triethyl-amine (TEA, MW=101.19 g/mol, ≥99.5%), (3-aminopropyl)triethoxysilane (APTS, MW=221.37 g/mol, ≥99%) and Dimethyl sulfoxide (DMSO, anhydrous, MW=78.13 g/mol, ≥99.9%), dimethylformamide (DMF) and diamine (i-iv) were purchased from Sigma-Aldrich. 3-isocyanatopropyltriethoxysilane (MW=237.36 g/mol, ≥95%), and 2-[methoxy(polyethyleneoxy)₆₋₉propyl]trimethoxysilane (MW_{avg}=525 g/mol, ≥90%) were purchased from Gelest. 2.8 μm beads coated (Dynabeads beads) with streptavidin were purchase by ThermoFisher scientific and antibody labelled with biotin and Ru(bpy)₃²⁺.

Synthesis

Synthesis of silica precursor/s of the silicate network core of the nanoparticle according to the present invention, such as alkoxysilane precursors

Synthesis—¹H NMR Characterization of Some Organosilane/Alkoxysilane Precursors, Such as Organoethoxysilane Derivatives 1-4

The organoethoxysilane derivatives 1-4 were synthesized by click reactions between the corresponding diamine (i-iv) and (3-isocyanatopropyl)triethoxysilane. In a typical preparation, 0.2 mmol of a diamine were dissolved in 0.1 mL of dimethylformamide (DMF) and 0.4 mmol of (3-isocyanatopropyl)triethoxysilane were added. This mixture was vortexed for 1 minute, and then stirred for 30 minutes at room temperature. Each synthesis was performed prior the preparation of nanoparticles and their product used without further purification.

### 1,1′-(ethane-1,2-diyl)bis(3-(3-(triethoxysilyl)propyl)urea) (1)

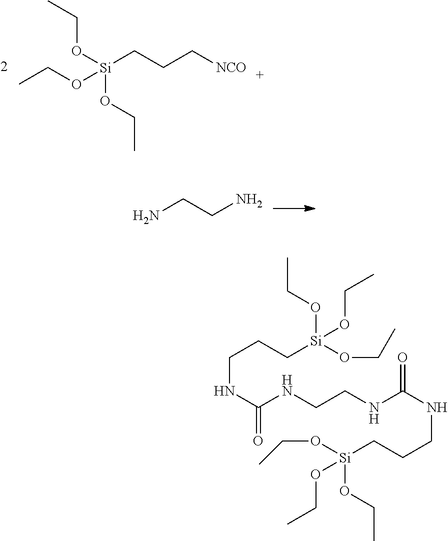

¹H NMR (400 MHz, DMSO-d8, 25° C.) δ (ppm): 0.51 (m, 4H, —NHCH₂CH₂CH₂—Si), 1.14 (t, J 8.0 Hz, 18H, —Si(OCH₂CH₃)₃), 1.41 (m, 4H, —NHCH₂CH₂CH₂—Si), 2.94 (q, 4H, J 4.0 Hz, —NHCH₂CH₂CH₂—Si), 2.99 (t, J 4.0 Hz, 4H, —NHCH₂CH₂NH—), 3.74 (q, J 8.0 Hz, 12H, —Si—(OCH₂CH₃)₃), 5.86 (t (broad), J 8.0 Hz, 2H, —NHCH₂CH₂CH₂—Si), 5.96 (t (broad), J 8.0 Hz, 2H, —NHCH₂CH₂NH—).

¹³C NMR (75.5 MHz, DMSO-d8, 25° C.) ä (ppm): 7.3, 18.2, 22.6, 40.1, 42.1, 57.7, 158.2.

### 1,1′-(hexane-1,6-diyl)bis(3-(3-(triethoxysilyl)propyl)urea) (2)

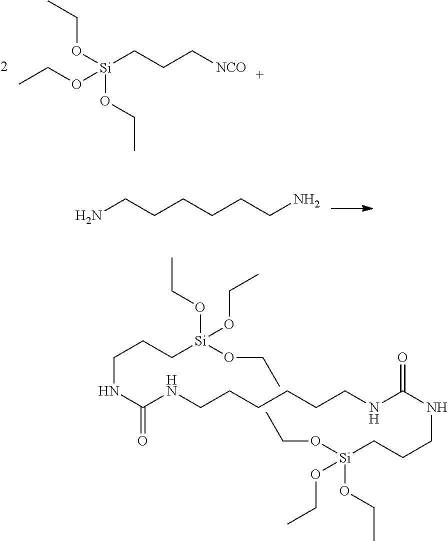

¹H NMR (600 MHz, DMSO-d8, 40° C.) δ (ppm): 0.51 (m, 4H, —NHCH₂CH₂CH₂—Si), 1.15 (t, J 12.0 Hz, 18H, —Si(OCH₂CH₃)₃), 1.24 (m, 4H, —NHCH₂CH₂CH₂CH₂CH₂CH₂NH—), 1.34 (t (broad), J 6.0 Hz, 4H, —NHCH₂CH₂CH₂CH₂CH₂CH₂NH—), 1.41 (m, 4H, —NHCH₂CH₂CH₂—Si), 2.95 (m, 8H, CH₂NH—CO—NHCH₂CH₂CH₂—Si), 3.74 (q, J 6.0 Hz, 12H, —Si—(OCH₂CH₃)₃), 5.70 (t (broad), J 6.0 Hz, 2H, —NHCH₂CH₂CH₂—Si), 5.76 (t (broad), J 6.0 Hz, 2H, —NHCH₂CH₂CH₂CH₂CH₂CH₂NH—).

¹³C NMR (151 MHz, DMSO-d8, 40° C.) δ (ppm): 7.2, 18.0, 23.5, 26.1, 30.0, 39.1, 41.9, 57.6, 158.

### 1,1′-(1,4-phenylene)bis(3-(3-(triethoxysilyl)propyl)urea) (3)

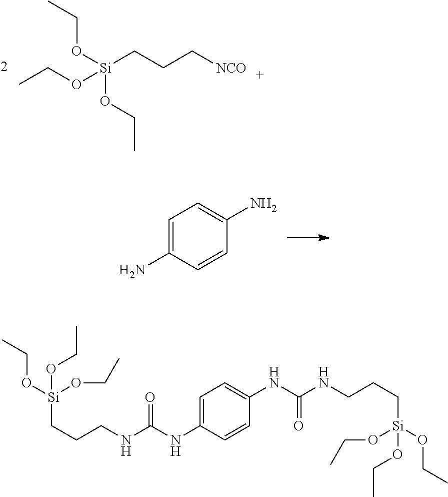

¹H NMR (400 MHz, DMSO-d8, 25° C.) δ (ppm): 0.53 (m, 4H, —NHCH₂CH₂CH₂—Si), 1.15 (t, J 8.0 Hz, 18H, —Si(OCH₂CH₃)₃), 1.47 (m, 4H, —NHCH₂CH₂CH₂—Si), 3.03 (m, 4H, —NHCH₂CH₂CH₂—Si), 3.74 (q, J 8.0 Hz, 12H, —Si(OCH₂CH₃)₃), 6.04 (t (broad), J 4.0 Hz, 2H, —NHCH₂CH₂CH₂—Si), 7.22 (s, 4H, Harom), 8.14 (s, 2H, —CONH—C₆H₆—NHCO—)

¹³C NMR (75.5 MHz, DMSO-d8, 25° C.) å (ppm): 7.3, 18.2, 23.5, 41.8, 57.7, 114.2, 134.4, 155.4.

### 1,1′-(ethane-1,2-diyl)bis(1-phenyl-3-(3-(triethoxysilyl)propyl)urea) (4)

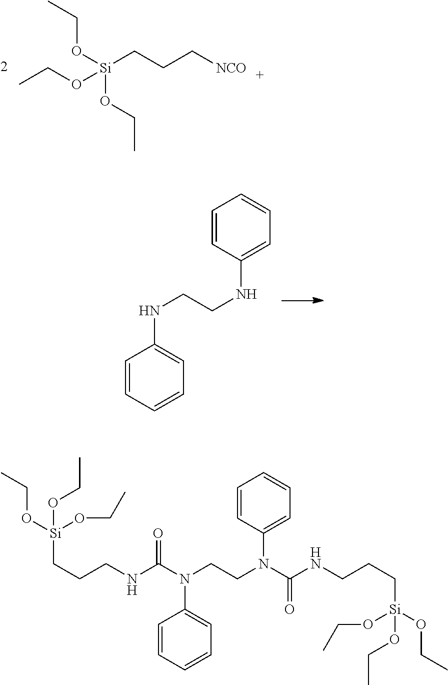

¹H NMR (400 MHz, DMSO-d8, 25° C.) δ (ppm): 0.61 (m, 4H, —NHCH₂CH₂CH₂—Si), 1.17 (t, J 8.0 Hz, 18H, —Si(OCH₂CH₃)₃), 1.62 (m, 4H, —NHCH₂CH₂CH₂—Si), 3.23 (s (broad), 4H, —NHCH₂CH₂NH—), 3.31 (t, 4H, J 4.0 Hz, —NHCH₂CH₂CH₂—Si), 3.77 (q, J 8.0 Hz, 12H, —Si—(OCH₂CH₃)₃), 5.58 (s (broad), 2H, —NHCH₂CH₂CH₂—Si), 6.54 (m, 2H), 6.60 (m, 4H), 7.08 (m, 2H) Hₐᵣₒₘ

¹³C NMR (75.5 MHz, DMSO-d8, 25° C.) δ (ppm): 7.0, 18.1, 24.6, 42.1, 44.9, 57.7, 58.1, 112.0, 116.2, 128.8, 148.7.

Synthesis of Ru(bpy)₃²⁺-TES derivative. This compound (FIG. 1B) was obtained by synthesizing and coupling Bis(2,2′-bipyridine)-[4-(4′-methyl-2,2′-bipyridin4yl) aminobutyl]ruthenium(II) bis(hexafluorophosphate), and 3-isocyanatopropyltriethoxysilane according to previously reported procedures (see scheme S1)

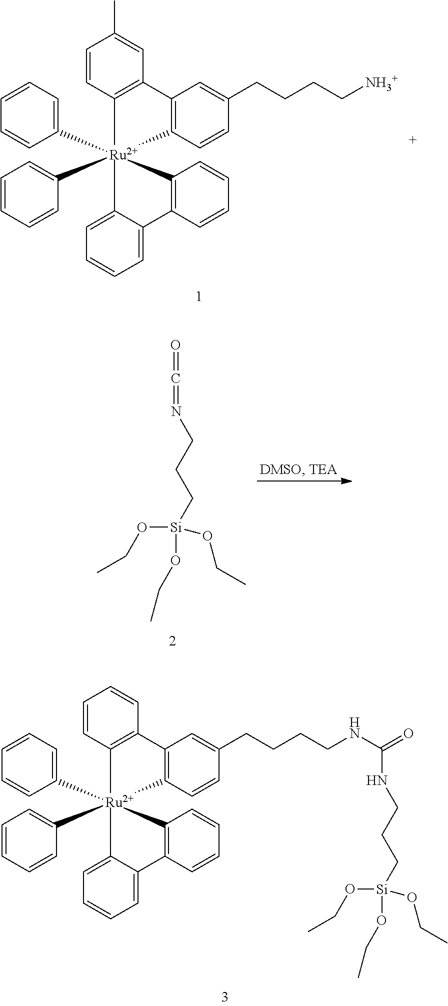

Scheme S1. Synthetic scheme of Ru(bpy)₃²⁺-TES derivative (3) obtained by coupling Bis(2,2′-bipyridine)-[4-(4′-methyl-2,2′-bipyridin4yl)aminobutyl]ruthenium(II) is (hexafluorophosphate) (1) and 3-isocyanatopropyltriethoxysilane (2).

Synthesis of Biotin-PEG45-TES derivative.

The synthesis of the PEG derivative started dissolving in a 1.5 ml plastic tube, about 5 mg of Biotin-PEG45-NHS (1.66 μmol) with 100 μL of DMSO and then adding 0.5 μL of APTES (2.12 μmol).

The reaction is kept under mixing with a vortex, for 2 h and then used without any further purification for the surface functionalization of the silica nanoparticles.

Preparation of Covalently Doped Ru(bpy)₃²⁺ Triton or Igepal Silica Nanoparticles. The synthetic scheme of the preparation of Dye Doped Silica Nanoparticles (DDSNPs), is shown in FIG. 1.

Two families of DDSNPs were synthesized by using a reverse microemulsion method (RME) at room temperature. Surfactant (Triton X-100 or Igepal CO-520), cyclohexane, water and TEOS were introduced in a glass vial under rapid magnetic stirring creating a ternary microemulsion system.

For silica nanoparticles using Triton X-100 as a surfactant, n-hexanol was also added to the quaternary microemulsion.

After a 20 min equilibration time Ru(bpy)₃²⁺-TES derivative was added to the mixture; after mixing for 20 minutes aqueous ammonia NH₄OH was added as both a reactant (H₂O) and a catalyst (NH₃) for the hydrolysis of TEOS. The reaction was stirred for an additional 24 hours at room temperature, followed by the addition of PEG and silane-PEG-biotin for particle post-coating and surface modification.

The mixture was further reacted for an additional 24 hours with stirring.

Then the nanoparticles were isolated from the microemulsion using acetone, centrifugated/centrifuged multiple times at 4000 rpm for 3 min and washed with ethanol and water several times to remove any surfactant molecules. Ultrasonication was used during the washing process to remove any physically adsorbed fluorophores from the particle surfaces.

All the reagents employed with their exact quantities for the synthesis of bio-Triton@RuNP and bio-Igepal@RuNP are listed in Table 5.

TABLE 5

| | bio- | bio- |
|---|---|---|
| | Triton@RuNP | Igepal@RuNP |
| Triton X-100 [g] | 7.48 | — |
| Igepal CO-520 [g] | — | 5.76 |
| Cyclohexane [mL] | 30 | 15 |
| n-hexanol [mL] | 7 | — |
| H₂O [μL] | 1920 | 1 |
| TEOS [μL] | 400 | 123 |
| NH₄OH [μL] | 240 | 36 |
| Ru(bpy)₃²⁺-TES [mg] | 32.3 | 9.9 |
| DAY 2 (after 24 h) | | |
| PEG 6-9 [μL] | 5500 | 1390 |
| PEG-biotin [mg] | 5 | 6 |

Beads Functionalization

To enable the ECL imaging measurements, streptavidin-coated beads with a diameter of 2.8 μm were functionalized with silica nanoparticle/s or dye-doped silica nanoparticle/s (DDSNP/s) such as bio-Triton@RuNps. The magnetic beads solution (diameter 2.8 μm; Dynabeads beads (ThermoFisher scientific) 6 mL, (total surface area of 7×10⁹ μm²) was poured in a 20 mL vial, and beads were collected using a magnet for 2 minutes. Afterwards the supernatant was discharged and 18 mL of bio-Triton@RuNP solution (10 nM) in phosphate buffer (0.01 M) was added, followed with 2 hours incubation at 37° C. under rotation to form the biotin-streptavidin bond. The solution was separated with a magnet and the supernatant discharged. The whole procedure was repeated five times. At the end of the fifth cycle, beads@Triton were washed five times in phosphate buffer (0.2 M) and polidodecanol surfactant to eliminate the unbounded nanoparticles and stored in bead buffer at 4° C. Beads@bio-Ru were obtained using the same procedure but using Free Conjugate (antibody labelled with biotin and Ru(bpy)₃²⁺) instead of bio-Triton@RuNps solution.

Photophysical Measurements

UV-Vis absorption spectra were recorded at 25° C. using a PerkinElmer Lambda 45 spectrophotometer. The fluorescence spectra were recorded with a PerkinElmer Lambda LS55 fluorimeter and with a modular UV-Vis-NIR spectrofluorimeter Edinburgh Instruments FLS920 equipped with a photomultiplier HamamatsuR928P. The latter instrument connected to a PCS900 PC card was used for the time-correlated single-photon counting (TCSPC) experiments (excitation laser A=410 nm). NPs suspension were diluted with milli-Q water. Luminescence quantum yields (uncertainty ±15%) were recorded on air-equilibrated water solutions using Ru(bpy)₃²⁺ as reference dye.^{[4]} The phosphorescence lifetime decays are fitted with a bi-exponential decay, the lifetimes values are reported as a weighted mean of two fitted components.

Transmission electron microscopy (TEM) and Dynamic Light Scattering (DLS).

For TEM investigations, a Philip CM 100 transmission electron microscope was used operating at 60 KV and 3.05 mm copper grids (Formvar support film—400 mesh). A drop of DDSNs solution diluted with water (1:50) was placed on the grid and then dried under a vacuum. The TEM images showing the denser silica cores were analysed with the ImageJ software, considering a few hundred nanoparticles. The obtained histogram was fitted according to a Gaussian distribution obtaining the average diameter for the silica nanoparticles.

Silica nanoparticles size distributions of the silica nanoparticle according to the present invention were determined by dynamic light scattering (DLS) employing a Malvern Nano ZS instrument with a 633 nm laser diode. Samples were housed in disposable polystyrene cuvettes of 1 cm optical path length. The width of the DLS hydrodynamic diameter distribution is indicated by the PdI (polydispersion index). In the case of a monomodal distribution (Gaussian) calculated using cumulant analysis PdI=(σ/Zavg)², where σ is the width of the distribution and Z_{avg} is average diameter of the particles population respectively.

ζ-Potential Experiments

ζ-potential values were determined using a Malvern Nano ZS instrument. Samples were housed in disposable polycarbonate folded capillary cell (DTS1070, 750 μL, 4 mm optical path length). Electrophoretic determination of (potential was made under Smoluchowski approximation in aqueous media at moderate electrolyte concentration.

Laser Scanning Confocal Fluorescence and Fluorescence Lifetime Imaging (FLIM)

The functionalized beads were characterized with an inverted Nikon A1R laser scanning confocal microscope. Images were collected using a Nikon PLAN APO 100× oil immersion objective, NA 1.45. Pinhole was set to 1 Airy Unit. In Laser Scanning Confocal Fluorescence Imaging a 401 nm CW laser was used as excitation, which was reflected onto a dichroic mirror (405 nm), while emission photons were collected through a 595/50 nm emission filter. In FLIM a time-correlated single photon counting (TCSPC) system of Picoquant GmbH Berlin was used with a 405 nm pulsed excitation laser at 25 kHz repetition frequency, the same dichroic mirror, a 560 nm long-pass emission filter, a Hybrid PMA detector and a Picoquant TimeHarp correlation board.

Estimation of the Average Number of the Complexes Inside Each NP

The weight of each NPs has been obtained calculating the volume of the NPs from the core diameter measured by TEM images and taking 2.0 g mL⁻¹ as the density of the silica matrix.

From this value, the number of NPs produced during the synthetic step was estimated assuming that all the TEOS introduced were converted in silica NPs, an assumption that has been found valid if a sufficient time is allocated before NPs isolation, as in this case. The final concentration has been calculated knowing the volume of water added to prepare the solution form isolated NPs.

From the absorption spectra has been finally possible to determine the total concentration of the Ru complexes, assuming the same molar excitation coefficient for the complex in solution (at 452 nm ε=14600 cm⁻¹ M⁻¹) as embedded in the silica lattice; and dividing this value by the concentration of the NPs is possible to estimate the average number of Ru complexes contained in each NP.

Inductively Coupled Plasma Mass Spectrometry

X Series^{II} ICP-MS from Thermo Fisher was used to quantify the Ru conjugated to beads (beads@Triton and beads@bio-Ru). Briefly, 500 μL of beads was dissolved in 358 μL of nitric acid (70%) and double-distilled water at a final volume of 5 mL and incubated overnight at 80° C. After dissolution, a clear solution was obtained. The total amount of Ru, as ppb concentration, was normalized to the total surface area of each bead size to obtain the density Ru μm⁻² (see Table 4).

ECL and ECL Imaging Measurements

ECL measurements were carried out with PGSTAT30 Ecochemie AUTOLAB electrochemical station in a three electrodes home-made transparent plexiglass cell using a glassy carbon (GC) 2 mm diameter disk as working electrode, a Pt spiral as counter electrode and Ag/AgCl, KCl (3 M) as reference electrode. ECL measurements were performed on NPs suspension diluted with phosphate buffer (PB, pH 7.4). For ECL generation, 180 mM TPrA was used as oxidative co-reactant. The ECL signal generated by performing the potential step programs was measured with a photomultiplier tube Acton PMT PD471 placed at a constant distance in front of the cell and inside a dark box. A voltage of 750 V was supplied to the PMT. The light/current/voltage curves were recorded by collecting the pre-amplified PMT output signal (by an ultralow-noise Acton research model 181) with the second input channel of the ADC module of the AUTOLAB instrument.

The ECL/optical imaging of beads@Triton and beads@bio-Ru, deposited on the working electrode and collected by a magnet, was performed using a solution of 0.2 M PB (pH 6.9), 180 mM TPrA and polidodecanol surfactant in a PTFE homemade electrochemical cell comprising Pt working (0.16 cm²), Pt counter, and Ag/AgCl (3 M KCl) reference electrodes. For microscopic imaging, an epifluorescence microscope from Nikon (Chiyoda, Tokyo, Japan) equipped with an ultrasensitive EMCCD camera (EM-CCD 9100-13 from Hamamatsu, Hamamatsu Japan) was used with a resolution of 512×512 pixels and a size of 16×16 pmt. The microscope was enclosed in a homemade dark box to avoid interferences from external light. It was also equipped with a motorized microscope stage (Corvus, Marzhauser, Wetzlar, Germany) for sample positioning and with long-distance objectives from Nikon (100×/0.80/DL4.5 mm and 40×/0.60/DL3.6). The integrated system also included a potentiostat from AUTOLAB (PGSTAT 30). Images were recorded during the application of a constant potential of 1.4 V (vs. Ag/AgCl 3M KCl) for 4 s with an integration time of 8 s.

1. A silica nanoparticle comprising:
a) a silicate network core with a diameter in a range from 10 nm to 500 nm, wherein at least a luminophore (dye) is confined in the said silicate network core, or covalently linked to the silicate network of the core because said luminophore (dye) has an anchoring moiety able to form at least a covalent link with the silicate network core, and
b) a shell layer over the silicate network core incorporating dye, said shell layer having a thickness from 0.5 to 7 nm, and comprising a colloidal stabilizer agent, selected from the group comprising: sterical or electrostatic antifouling agents or polyether antifouling agents.

2. The nanoparticle according to claim 1, wherein the anchoring moiety is one of a hydrophobic anchoring moiety, an alkoxysilane moiety, and a trialkoxysilane moiety.

3. The nanoparticle according to claim 1, wherein the shell layer comprises a bio-linker (bl) and/or a chemical-linker (cl) and/or a biochemical-linker (bcl) each of them comprising an anchoring moiety bl-1), cl-1) or bcl-1), respectively, onward/projecting towards the silicate network core incorporating dye and a hydrophilic moiety bl-2), cl-2) or bcl-2), respectively, outward/projecting outwards from the silicate network core incorporating dye, wherein:
a terminal part of the hydrophilic moiety bl-2), cl-2) or bcl-2), respectively, comprises at least a biorecognition moiety br-3), a chemical recognition moiety cr-3) or biochemical recognition moiety bcr-3), respectively, or
a terminal part of the hydrophilic moiety bl-2), cl-2) or bcl-2), respectively, comprises at least one functional group, useful for the introduction of a biorecognition moiety br-3), a chemical recognition moiety cr-3) or biochemical recognition moiety bcr-3),
respectively, said functional group preferably selected from the group comprising amine, —COOH, —N₃, alkyne, alkene, acryloyl, —SH, maleimide, aldehyde, —OH, isothiocyanate, sulfonyl chloride, iodoacetyl, TCT (2,4,6-Trichloro-1,3,5-triazine) or an activated carboxylic group such as NHS and NHS-sulfo esters (N-hydroxysuccinimide and sulfo N-hydroxysuccinimide), TFP ester (2,3,5,6-Tetrafluorophenol), PFP ester (pentafluorophenol), HOBt ester (1-hydroxybenzotriazole), N-acylimidazole.

4. The nanoparticle according to claim 1, wherein the luminophore (dye) is a luminophore (dye) having a functionality useful for the introduction of an anchoring moiety, including one of a hydrophobic anchoring moiety, an alkoxysilane moiety, and a trialkoxysilane moiety.

5. The nanoparticle according to claim 4, wherein said functionality is selected from the group comprising amine, —COOH, —N₃, alkyne, alkene, acryloyl, —SH, maleimide, aldehyde, —OH, isothiocyanate, sulfonyl chloride, iodoacetyl, TCT (2,4,6-Trichloro-1,3,5-triazine) or an activated carboxylic group such as NHS and NHS-sulfo esters (N-hydroxysuccinimide and sulfo N-hydroxysuccinimide), TFP ester (2,3,5,6-Tetrafluorophenol), PFP ester (pentafluorophenol), HOBt ester (1-hydroxybenzotriazole), N-acylimidazole.

6. The nanoparticle according to claim 1, wherein the luminophore (dye) is: a metal complex, more preferably selected from the group comprising:
Ruthenium(II) polypyridine derivatives, such as Ru(bpy)₃²⁺ where bpy is tris(2,2′-bipyridine), Ru(phen)₃²⁺ where phen is 1,10-Phenanthroline, or Ru(bpy)₂ (bps)—where bps is 4,7-diphenyl-1,10-phenanthroline disulfonate, and derivates thereof with general structure [Ru(bpy)₃ (bps)]²⁻²ⁿ or [Ru(phen)₃₋ₙ(bps)ₙ]²⁻²ⁿ wherein n is 1, 2 or 3, Ru(phen)₂ (dppz)′ or Ru(bpy)₂ (dppz)²⁺—where dppz is dipyrido[3,2-a: 2,3-c]phenazine,
Cyclometalated Ir(III) metal complexes homoleptic, such us Ir(C\hat{ }N)₃ where CAN is a monoanionic ligand such as 2-Phenylpyridine, and heteroleptic Ir(C\hat{ }N)₂ (L\hat{ }L) where C\hat{ }N is a monoanionic ligand such as 2-Phenylpyridine and LAL is 2,2′-bipyridine, or
an organic luminophore, more preferably selected from the group comprising: anthracene derivatives, xanthene dyes derivatives, cyanine derivatives, bodipy dye derivatives and coumarin dye derivatives.

7. The nanoparticle according to claim 1, wherein the colloidal stabilizer agent of the shell layer is a colloidal stabilizer agent, including sterical or electrostatic antifouling agents or polyether antifouling agents, the colloidal a stabilizer agent comprising at least a chain structure (cs) having an anchoring moiety cs-1) onward/projecting towards the silicate network core incorporating dye and a hydrophilic moiety cs-2) outward/projecting outwards from the silicate network core incorporating dye, wherein the anchoring moiety cs-1) is one of a hydrophobic anchoring moiety, an alkoxylate moiety, a trialkoxysilane moiety and/or the hydrophilic moiety cs-2) comprises a polyether moiety, including a PEGn-OH [-Poly(ethylene glycol)n-OH] moiety wherein n is from 3 to 100.

8. A diagnostic probe comprising:
a silica nanoparticle comprising:
a) a silicate network core with a diameter in a range from 10 nm to 500 nm, wherein at least a luminophore (dye) is confined in the said silicate network core, or covalently linked to the silicate network of the core because said luminophore (dye) has an anchoring moiety able to form at least a covalent link with the silicate network core, and
b) a shell layer over the silicate network core incorporating dye, said shell layer with a thickness from 0.5 to 7 nm, comprising a colloidal stabilizer agent, selected from the group comprising: sterical or electrostatic antifouling agents or polyether antifouling agents.

9. An-analytical chemical probe comprising:
a silica nanoparticle comprising:
a) a silicate network core with a diameter in a range from 10 nm to 500 nm, wherein at least a luminophore (dye) is confined in the said silicate network core, or covalently linked to the silicate network of the core because said luminophore (dye) has an anchoring moiety able to form at least a covalent link with the silicate network core, and
b) a shell layer over the silicate network core incorporating dye, said shell layer with a thickness from 0.5 to 7 nm, comprising a colloidal stabilizer agent, selected from the group comprising: sterical or electrostatic antifouling agents or polyether antifouling agents.

10. A diagnostic composition comprising an amount of a silica nanoparticle comprising:
a) a silicate network core with a diameter in a range from 10 nm to 500 nm, wherein at least a luminophore (dye) is confined in the said silicate network core, or covalently linked to the silicate network of the core because said luminophore (dye) has an anchoring moiety able to form at least a covalent link with the silicate network core, and
b) a shell layer over the silicate network core incorporating dye, said shell layer with a thickness from 0.5 to 7 nm, comprising a colloidal stabilizer agent, selected from the group comprising: sterical or electrostatic antifouling agents or polyether antifouling agents.

11. A process for manufacturing a nanoparticle comprising the Reverse Micro-Emulsion (RME) method and comprising the steps of:
i) preparing a water-in-oil emulsion stabilized with non-ionic surfactant/s, and eventually by the presence of co-surfactant/s,
ii) adding to the water portion of the stabilized water-in-oil emulsion obtained in step i) a luminophore having an anchoring moiety, a silica precursor and a base to form/to obtain a silicate network core incorporating dye of the nanoparticle, wherein the silicate network core has a diameter in a range from 10 nm to 500 nm, wherein at least the luminophore (dye) is confined in the said silicate network core, or covalently linked to the silicate network of the core because said luminophore (dye) has an anchoring moiety able to form at least a covalent link with the silicate network core, and iii) coating the silicate network core a) incorporating dye/s obtained in step ii) by adding a colloidal stabilizer agent, to form/to obtain a shell layer of the nanoparticle, wherein the a shell layer is formed over the silicate network core incorporating dye, said shell layer having a thickness from 0.5 to 7 nm, and comprising a colloidal stabilizer agent, selected from the group comprising: sterical or electrostatic antifouling agents or polyether antifouling agents.

12. The process according to claim 11, wherein in step iii) it is also added a linker selected from the group comprising: bio-linker and/or biochemical-linker and/or chemical-linker.

13. The process according to claim 11, further comprising the step of:
iv) purifying the nanoparticle obtained in step iii) from non-ionic surfactant and oil.

14. The nanoparticle according to claim 1, wherein the silicate network core diameter is in a range from 20 nm to 250 nm.

15. The nanoparticle according to claim 1, wherein the shell layer thickness is from 0.5 to 6 nm.

16. The nanoparticle according to claim 1, wherein the shell layer thickness is from 1 to 5 nm.
